(19) United States
(12) Patent Application Publication
(10) Pub. No.: US 2005/0177125 A1
(43) Pub. Date: Aug. 11, 2005

(54) Pant-type disposable garment

(76) Inventor: Masahiro Kondo, Kobe (JP)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/051,813

(22) Filed: Feb. 4, 2005

Related U.S. Application Data
(60) Provisional application No. 60/543,398, filed on Feb. 10, 2004.

(51) Int. Cl. A61F 13/15; A61F 13/20

(52) U.S. Cl. 604/385.29; 604/394

(57) ABSTRACT

A pant-type disposable garment having a waist opening and a pair of leg openings when in use of the garment is disclosed. The garment comprises a first waist panel, a second waist panel and a crotch panel positioned between the first waist panel and the second waist panel. Each of the first and the second waist panels has an interior surface orienting the wearer's skin and an exterior surface. The crotch panel comprises a first end portion joined to the first waist panel when in use of the garment and a second end portion joined to the second waist panel when in use of the garment. The first end portion of the crotch panel is positioned inside the first waist panel and the second end portion of the crotch panel is positioned inside the second waist panel before use of the garment. The second end portion of the crotch panel is capable of being joined to the exterior surface of the second waist panel to form a pant shape of the garment after the second end portion of the crotch panel is displaced from the inside of the second waist panel.

### CROSS REFERENCE TO RELATED APPLICATION

This application claims the benefit of U.S. Provisional Application No. 60/543398, filed Feb. 10, 2004.

### FIELD OF THE INVENTION

The present invention relates to a pant type disposable garment having a refastenable fastening system used for infants, adults or incontinence persons; or a pant type disposable incontinence garment having a refastenable fastening system used with another absorbent article such as a supplemental inner pad.

### BACKGROUND OF THE INVENTION

A disposable absorbent article worn to assist in the collection of bodily discharges of incontinent persons is well known in the art. Such a conventional art includes a pant-type disposable diaper in which the ventral and dorsal portions of the diaper are connected and fixed together at both side edges of the ventral and dorsal portions to form a pair of leg openings and a unitary waist opening. For example, U.S. Pat. No. 5,449,353 and U.S. Pat. No. 5,735,839 disclose such a conventional pant-type diaper. One drawback with such a pant-type diaper is that the manner of applying the diaper is limited to being pulled on like a pant. Applying the absorbent article like a pant is advantageous in many cases, and is particularly suited for active, walking incontinent persons. However, even for the same wearer, there may be time when it would be useful to apply the absorbent article like a conventional tape-type diaper. For example, it might be more convenient to apply the absorbent article like a tape-type diaper when there is a desire not to remove the wearer's shoes. Since it is difficult to know when a particular mode of applying the absorbent article will be needed, it is beneficial to have an absorbent article that is adaptable to being used both as a pant-type diaper and as a tape-type diaper. This is preferable to keeping both types of absorbent article available in advance. An absorbent article that can be applied both like a pant-type diaper and like a tape-type diaper also permits the interior of the absorbent article to be easily checked without pulling the article downward.

A typical example of such a convertible pant-type diaper is disclosed in, e.g., International Patent Publications WO 00/35398. It discloses a pant-type disposable diaper in which the front and rear waist regions of the diaper are releasably fastened to each other at the side-panel portions of the diaper which are positioned on the hips of the wearer when the diaper is worn. The diaper includes a refastenable fastening system disposed to the side panels of the diaper for fastening the front and rear waist regions to each other. Such a disposable diaper as disclosed in WO 00/35398 can be applied both like a pant-type diaper and like a tape-type diaper because of the refastenable fastening system typically comprising a hook-type fastener and a loop-type fastener releasably engaged with the hook-type fastener. Such releasable fastening as disclosed in the above prior art is typically achieved by overlapping the side-panel portions of the front and rear waist regions with each other. The overlap of the side-panel portions of the front and rear waist regions forms undesirable bumps at the side-panel portions while the diaper is worn. Such an undesirable bump could easily trigger undesirable detachment of the overlapped side-panel portions from each other during use of the diaper, e.g., due to friction exerted between the diaper and the wearer's garment. Further, the side-panel portion of a convertible pant-type diaper is typically elasticized in the transverse direction of the diaper. Therefore, a user of such a conventional convertible pant-type diaper suffers from the drawback that it is difficult for the user to overlap the side-panel portions of the front and rear waist regions with each other. The drawback results from the fact that the transversely elongated side-panel portion tends to recover its original size/shape due to the elasticity of the side-panel portion.

Another example of a pant-type diaper is disclosed in Japanese Patent Laid-Open publication 2000-27003 and European Patent publication EP1133966A2. They disclose a disposable diaper in which the crotch portion of the diaper is openable and reclosable with respect to the interior surface of the front waist portion of the diaper. However, it is difficult for the user to reclose the crotch portion with respect to the front waist portion because the crotch portion must be joined to the interior surface of the front waist portion. Japanese Patent Laid-Open publication H09-566 discloses a disposable diaper in which the crotch portion of the diaper is openable and reclosable with respect to the exterior surface of the front waist portion of the diaper. However, such a diaper requires a complicated manufacturing process. In order to place the crotch panel under the exterior surface of the front waist portion in the manufacturing process, the crotch panel together with the back waist portion joined to the crotch panel must be fed precedent to the front waist portion and then the front waist portion is fed onto the crotch panel. Since the crotch panel is positioned underneath the waist portion, a supporting mechanism for supporting the crotch panel is required so that the crotch panel does not droop while the crotch panel is conveyed in the manufacturing process.

Based on the foregoing, there is a need for a pant-type disposable garment which can be formed with an easy manufacturing process while allowing easy operation of the openable crotch panel when in use of the garment.

### SUMMARY OF THE INVENTION

The present invention is directed to a pant-type disposable garment having a waist opening and a pair of leg openings when in use of the garment. The garment comprises a first waist panel, a second waist panel and a crotch panel positioned between the first waist panel and the second waist panel. Each of the first and the second waist panels has an interior surface orienting the wearer's skin and an exterior surface. The crotch panel comprises a first end portion joined to the first waist panel when in use of the garment and a second end portion joined to the second waist panel when in use of the garment. The first end portion of the crotch panel is positioned inside the first waist panel and the second end portion of the crotch panel is positioned inside the second waist panel before use of the garment. The second end portion of the crotch panel is capable of being joined to the exterior surface of the second waist panel to form a pant shape of the garment after the second end portion of the crotch panel is displaced from the inside of the second waist panel.

### BRIEF DESCRIPTION OF THE DRAWINGS

While the Specification concludes with claims which particularly point out and distinctly claim the invention, it is believed the present invention will be better understood from the following description of preferred embodiments taken in conjunction with the accompanying drawings, in which like reference numerals identify identical elements and wherein:

FIG. 1 is a top plan view of one embodiment of a garment of the present invention, and showing the wearer-facing surface of the garment;

FIG. 2 is a schematic perspective view of the garment illustrated in FIG. 1 before the releasable joint is released, and showing the first waist region of the garment;

FIG. 3 is a schematic perspective view of the garment illustrated in FIG. 1 before the releasable joint is released, and showing the second waist region of the garment;

FIG. 4 is a schematic perspective view of the garment illustrated in FIG. 1 after the releasable joint is released;

FIG. 5 is a schematic perspective view of the garment illustrated in FIG. 1 when the crotch panel is refastened to the second waist panel;

FIG. 6 is a cross-sectional view taken along line VI-VI of FIG. 1;

FIG. 7 is a cross-sectional view taken along line VII-VII of FIG. 1;

FIG. 8 is a schematic perspective view of one embodiment of the fastening member when the fastening member is permanently fixed to the crotch panel;

FIG. 9 is a schematic perspective view of the fastening member and the crotch panel before the fastening member is contained into a holder;

FIG. 10 is a schematic perspective view of the fastening member and the crotch panel when the fastening member is contained into a holder;

FIG. 11 is a schematic perspective view of another embodiment of a garment of the present invention before the releasable joint is released;

FIG. 12 is a schematic perspective view of the garment illustrated in FIG. 11 after the releasable joint is released;

FIG. 13 is a schematic perspective view of the garment illustrated in FIG. 11 when the crotch panel is refastened to the second waist panel;

FIG. 14 is a schematic top plane view of alternative embodiment of the garment of the present invention;

FIG. 15 is a schematic view of a process for forming the garment of the present invention;

FIG. 16 is a schematic perspective view of alternative embodiment of the garment before the releasable joint is released, and showing the second waist region of the garment;

FIG. 17 is a cross-sectional view taken along line XVII-XVII of FIG. 16;

FIG. 18 is a cross-sectional view in which the releasable joint is released and the second waist panel is removed from the configuration shown in FIG. 17;

FIG. 19 is a schematic perspective view of alternative embodiment of the garment of the present invention;

FIG. 20 is a cross-sectional view taken along line XX-XX of FIG. 19;

FIG. 21 is a cross-sectional view taken along line XXI-XXI of FIG. 19;

FIG. 22 is a cross-sectional view of another embodiment of FIG. 21;

FIG. 23 is a schematic perspective view of alternative embodiment of the garment of the present invention;

FIG. 24 is a cross-sectional view taken along line XXIV-XXIV of FIG. 23;

FIG. 25 is a schematic perspective view of the crotch panel of the garment shown in FIG. 23; and

FIG. 26 is a schematic perspective view of the garment of FIG. 23 when the crotch panel is refastened to the second waist panel.

### DETAILED DESCRIPTION OF THE INVENTION

The definitions of several terms are first provided to assist the reader in understanding the present invention.

The term “absorbent article(s)”, as used herein, refers to devices which absorb and contain body exudates, and more specifically, refers to devices which are placed against or in proximity to the body of the wearer to absorb and contain the various exudates discharged from the body. Such devices include disposable diapers, training pants, supplemental inner pads used with disposable diapers, sanitary napkins, adult/infant incontinent garments used with or without supplemental inner pads, and the like. The term “comprising” and its derivatives, as used herein, are intended to be open ended terms that specify the presence of the stated features, elements, components, groups, integers, and/or steps, but do not exclude the presence of other, unstated features, elements, components, groups, integers, and/or steps. This definition also applies to words of similar meaning, for example, the terms “have”, “include”, “be provided with” and their derivatives. The term “disposable”, as used herein, describes devices which generally are not intended to be laundered or otherwise restored or reused (i.e., they are intended to be discarded after a single use and, preferably, to be recycled, composted or otherwise disposed of in an environmentally compatible manner.) The terms “elastic” and “elastomeric” and their derivatives, as used herein, refer to a property of a material or a composite by virtue of which it tends to recover its original size and shape after removal of a force causing a deformation. The term “joined” or “joining”, as used herein, encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element, i.e. one element is essentially part of the other element. This definition also applies to words of similar meaning, for example, the terms “attached”, “bonded”, “fixed” and their derivatives. The term “member“when used in the singular can have the dual meaning of a single element or a plurality of elements. The term “nonwoven”, as used herein, refers to fabrics made of fibers held together by interlocking or bonding which are not woven, knitted, felted, or the like. The term “fabric”, as used herein, may refer to a nonwoven web, a woven material, or other types of fabrics. The term “permanently attached”, “permanently bonded”, “permanently fixed” or “permanently joined”, as used herein, refers to the joining, adhering, connecting, attaching, or the like, of two elements of an absorbent article such that the elements tend to be and remain bonded during normal use conditions of the garment. The term “pant-type garment”, “pull-on type garment” and variations thereof, as used herein, refer to a garment (e.g., a disposable garment) having fixed edges and leg openings. A pant-type garment is placed in position on the wearer by inserting the wearer's legs into the leg openings and sliding the pant-type article into position about the wearer's lower torso. The term “refastenable”, as used herein, refers to the property of two elements being capable of releasable attachment, separation, and subsequent releasable reattachment without substantial permanent deformation or rupture. The term “releasable”, as used herein, refers to the property of two elements being capable of at least separation. The terms “releasably joinable”, “releasably attachable”, “releasably engageable”, “releasably joined”, “releasably attached”, “releasably engaged” and variations thereof, as used herein, refer to two elements being connected or connectable such that the elements tend to remain connected in the absence of separation force applied to one or both of the elements, and the elements being capable of separation without substantial permanent deformation or rupture. The required separation force is typically beyond that encountered while wearing the absorbent article. The terms “releasably and repeatedly joined (joinable)” “releasably and repeatedly attached (attachable)”, “releasably and repeatedly engaged (engageable)” and variations thereof, as used herein, refer to two elements capable of separation without substantial permanent deformation or rupture, and capable of being reconnected or reconnectable many times after such separation.

The garment 100 typically comprises a liquid pervious topsheet, a liquid impervious backsheet, and an absorbent core positioned between the topsheet and the backsheet in order to acquire and store bodily discharges. Alternatively, any of the topsheet, the backsheet and the absorbent core may be omitted from the components of the garment 100 if the garment 100 is a garment intended to be used with another absorbent article such as a supplemental inner pad.

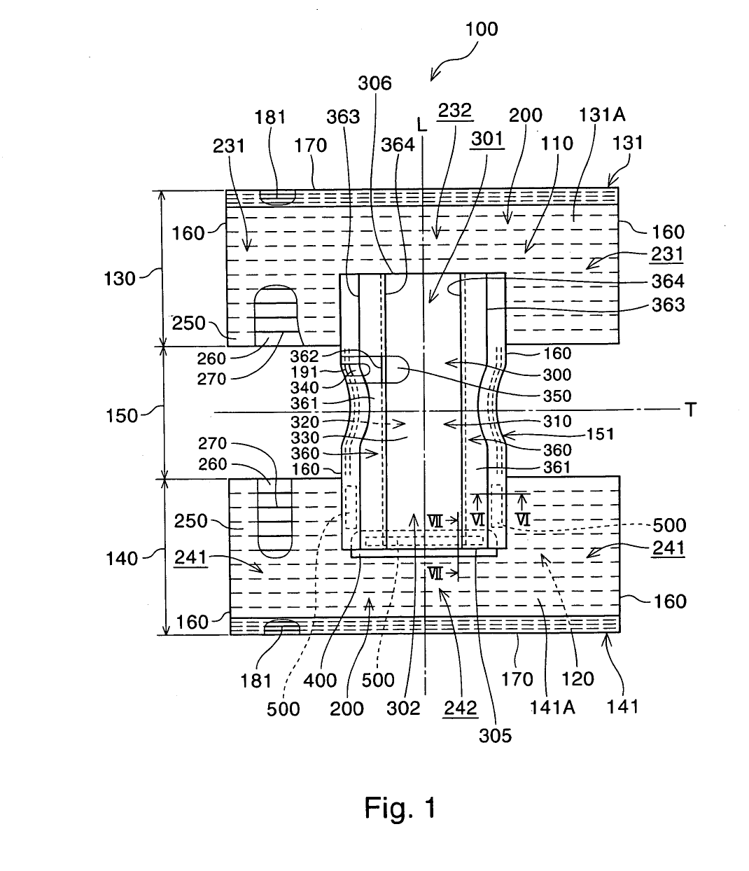

FIGS. 1 to 5 illustrate one embodiment of a pant-type disposable garment of the present invention suitable for use as, but not limited to, an incontinence aid. FIG. 1 illustrates a top plan view of a garment 100 according to the present invention, in its flat-out, uncontracted state (i.e., with elastic-induced contraction being pulled out) with portions of the structure being cut-away to show the underlying features. The present invention described herein is also applicable to a pant-type disposable garment such as a diaper cover or an incontinent garment used with a supplemental inner pad. As illustrated in FIG. 1, the garment 100 has a longitudinal centerline L. The term “longitudinal”, as used herein, refers to a line, axis or direction in the plane of the garment 100 that is generally aligned with (e.g., approximately parallel to) a vertical plane which bisects a standing wearer into left and right body halves when the garment 100 is worn. The garment 100 illustrated in FIG. 1 also has a transverse centerline T. The terms “transverse” or “lateral”, as used herein, are interchangeable, and refer to a line, axis or direction which lies within the plane of the garment 100 that is generally perpendicular to the longitudinal direction. The garment 100 has a periphery which is defined by side edges 160 and end edges 170. The garment 100 has two surfaces; one is a wearer-facing surface 110 and the other is an opposing surface 120. The wearer-facing surface 110 is the surface of the garment 100 which is generally oriented toward the wearer when the garment 100 is worn. The wearer-facing surface 110 typically at least partially comes in contact with the wearer's skin during use of the garment 100. The opposing surface 120 is the surface of the garment 100 which is generally oriented away from the wearer when the garment 100 is worn, and at least partially toward another garment if another garment is worn outside the garment 100. The garment 100 also has a first waist region 130, a second waist region 140 opposed to the first waist region 130, and a crotch region 150 positioned therebetween and connecting the first waist region 130 and the second waist region 140. The first waist region 130 and the second waist region 140 extend from the end edges 170 toward the crotch region 150. In one embodiment, the first and second waist regions 130, 140 may be the front and rear waist regions, respectively. Alternatively, the first and second waist regions 130, 140 may be the rear and front waist regions, respectively.

The garment 100 is configured to provide a pant configuration having a waist opening 180 and a pair of leg openings 190 at least when in use of the garment 100. The waist opening 180 is provided with one or more first elastic members 181 which at least partially, preferably entirely, form gathers along the circumference of the waist opening 180 for providing the fit of the garment 100 around the wearer's waist while the garment 100 is worn. Each leg opening 190 is provided with one or more second elastic members 191 which at least partially, preferably entirely, form gathers along the circumference of each leg opening 190 for providing the fit of the garment 100 around the wearer's leg while the garment 100 is worn.

The garment 100 comprises a first waist panel 131 positioned in the first waist region 130, a second waist panel 141 positioned in the second waist region 140 (the first and second waist panels may be collectively referred to as “waist panel”), and a crotch panel 151 positioned in the crotch region 150 and positioned between the first waist panel 131 and the second waist panel 141. In one embodiment, the first and second waist panels 131, 141 may be the front and rear waist panels, respectively. Alternatively, the first and second waist panels 131, 141 may be the rear and front waist panels, respectively. In the embodiment shown in FIGS. 1 to 5, the first waist panel 131 is the front waist panel and the second waist panel 141 is the rear waist panel. The crotch panel 151 is permanently joined to the first waist panel 131 by any known means such as adhesive, pressure bonding, heat bonding, etc., and is releasably joined to the second waist panel 141 by the releasable joint 500 which will be explained in detail hereinbelow. The crotch panel 151 is openable and reclosable with respect to the second waist panel 141. Alternatively, the crotch panel 151 may be releasably joined to both of the first waist panel 131 and the second waist panel 141 by releasable joints such that the crotch panel 151 may be openable and reclosable with respect to both of the first waist panel 131 and the second waist panel 141.

The garment 100 comprises a releasable joint 500 which releasably joins the crotch panel 151 to at least the interior surface 141A of the second waist panel 141 to preform a pant shape of the garment 100. When it is necessary to first open the crotch panel 151 from the second waist panel 141, the releasable joint 500 is released. The term “releasable joint”, as used herein, refers to a joint joining two elements to each other until intentional separation force is exerted on the joint while the joint is capable of being released without causing serious damage to the elements once intentional separation force is exerted on the joint. After the releasable joint 500 is first released, the releasable joint 500 may be incapable of joining the crotch panel 151 to the second waist panel 141. Alternatively, after the releasable joint 500 is first released, the releasable joint may be still capable of releasably joining the crotch panel 151 to the second waist panel 141. When the releasable joint 500 is released, the crotch panel 151 is openable from the second waist panel 141 such that the opening is provided for accessing the inside of the garment 100. Thus, the user can access the inside of the garment 100 to check the inside of the garment 100, to clean the inside of the garment 100, or to change the supplemental inner pad placed inside the garment 100 while the waist panel 131, 141 of the garment 100 is still anchored to the wearer. The crotch panel 151 must be reclosable with respect to the second waist panel 141 such that the garment 100 is reformed into a pant shape after the releasable joint 500 is released. For that purpose, the garment 100 further comprises a fastening member 400 being capable of refastening the crotch panel 151 to the exterior surface 141 B of the second waist panel 141. When it is necessary to reopen the crotch panel to, e.g., check the inside of the garment 100, the user/caregiver separates the crotch panel 151 from the second waist panel 141 by releasing the fastening member 400. After the operation is done for the inside of the garment 100, the user/caregiver releasably reattaches the crotch panel 151 to the exterior surface 141B of the second waist panel 141 with the fastening member 400 to reform a pant shape of the garment 100.

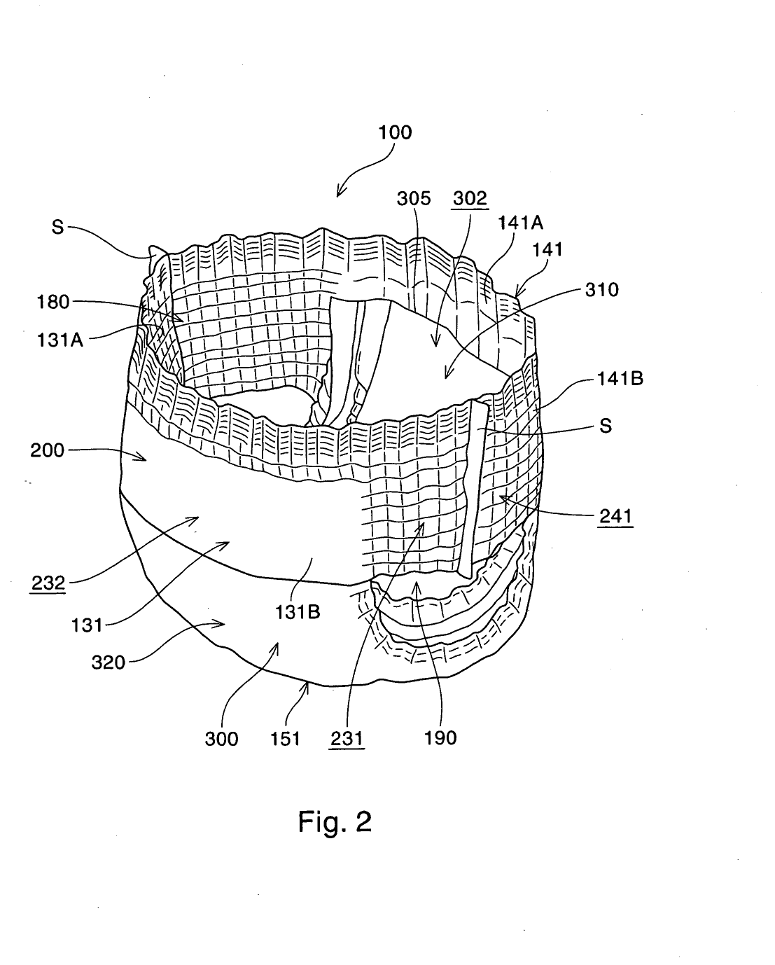

The waist panel 131, 141 encircles the waist of the wearer, and makes the crotch panel fit to the wearer's body while the garment 100 is worn. As illustrated in FIGS. 1 and 2, the waist panel 131, 141 has typically two surfaces; one is an interior surface 131A, 141A and the other is an exterior surface 131B, 141B. The interior surface 131A, 141A is the surface of the waist panel 131, 141 which is generally oriented toward the wearer when the garment 100 is worn. The interior surface 131A, 141A typically at least partially comes in contact with the wearer's skin during use of the garment 100. The exterior surface 131B, 141B is the surface of the waist panel 131, 141 which is generally oriented away from the wearer when the garment 100 is worn, and at least partially toward another garment if another garment is worn.

The waist panel 131, 141 preferably takes the form of belt as shown in FIG. 1. The first and second waist panels 131, 141 comprise a first waist belt member and a second waist belt member respectively and can be made from a unitary piece of material or from a number of separate pieces of material that may be identical or different. In the embodiment illustrated in FIGS. 1 to 5, the first waist panel 131 comprises two first side segments 231 adjacent to one of the side edges 160 and a first central segment 232 positioned between the first side segments 231. The first central segment 232 typically covers the ventral waist of the wearer when the garment 100 is worn. The second waist panel 141 comprises two second side segments 241 adjacent to one of the side edges 160 and a second central segment 242 positioned between the second side segments 241. The second central segment 242 typically covers the dorsal waist of the wearer when the garment 100 is worn. As illustrated in FIGS. 2 to 5, each first side segment 231 is permanently joined to each second side segment 241 at the side seams S to form a ring-like waist panel 200. Heat bonds, pressure bonds, ultrasonic bonds, or any other suitable means or combinations thereof as are known in the art can be used for bonding the first side segment 231 and the second side segment 241 to each other.

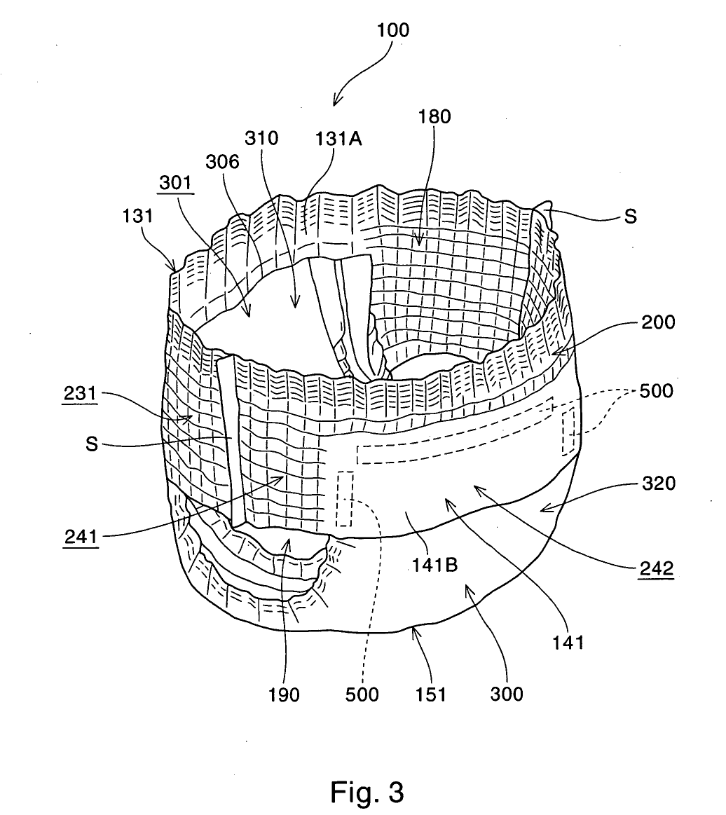

The waist panel 131, 141 may comprise one or multiple layers, preferably two or three layers. As illustrated in FIG. 1, the waist panel 131, 141 comprises two layers, which comprises an interior layer 250 and an exterior layer 260. The interior layer 250 is positioned on the interior surface 131A, 141A, and typically at least partially contacts with the wearer's skin when the garment 100 is worn. The exterior layer 260 is positioned on the exterior surface 131B, 141B of the waist panel 131, 141, and typically at least partially contacts with the garments of the wearer if another garment is worn. The waist panel 131, 141 can be elasticized by one or more elastic strands 270 disposed between the interior layer 250 and the exterior layer 260 for providing the fit of the waist panel 131, 141 around the wearer's torso while the garment 100 is worn. The elastic strands 270 allow the waist panel 131, 141 to elastically expand and contract such that the waist panel 131, 141 dynamically fits the wearer's body during use. The elastic strands 270 may be disposed only on the portion of the first and second waist panels 131, 141 with which the crotch panel 151 does not overlap (refer to FIGS. 2 and 3). Alternatively, the elastic strands 270 may extend into the portion of the first and second waist panels with which the crotch panel 151 overlaps.

The waist panel 131, 141 preferably comprises a nonwoven material. Such a nonwoven material for the waist panel 131, 141 is preferably air pervious. However, embodiments are contemplated wherein the waist panel 131, 141 comprises materials such as woven webs, scrims, films, loose fibers, or any other material or combination of materials known in the art that will give the waist panel 131, 141 a cloth-like look and/or feel and is at a minimum air permeable. If the waist panel 131, 141 comprises two layers, i.e., the interior layer 250 and the exterior layer 260, such layers may in particular comprise any material such as a nonwoven or a polymeric film. In a preferred embodiment, the layers may be formed from a laminate comprising two nonwoven layers, or a nonwoven layer and a polymeric film. The layer positioned on the interior surface 131A, 141A of the waist panel 131, 141 is preferably provided with a nonwoven layer. Such a nonwoven layer presents a compliant surface to the skin of a wearer and thus greatly improves skin healthiness. The waist panel 131, 141 may also comprise three layers; one film layer and two nonwoven layers. Preferably, the film may be interposed between the two nonwoven layers. This sequence of layers results in a closed fibrous structure, which has a particularly pleasing sensation on contact with the skin of a wearer.

Suitable nonwoven layers for the waist panel 131, 141 may comprise felt fabrics, spunlaced fabrics, fluid jet entangled fabrics, air-laid fabrics, wet-laid fabrics, dry-laid fabrics, melt-blown fabrics, staple fiber carding fabrics, spunbonded fabrics, stitch-bonded fabrics, apertured fabrics, combinations of the above or the like. The nonwoven layer or the nonwoven layers constituting the waist panel 131, 141 may be hydrophobic or hydrophilic. Suitable film materials for the waist panel 131, 141 may comprise a thermoplastic material. The thermoplastic material can be selected from among all types of polyolefins especially polyethylene, polypropylene, amorphous polyolefins, and the like; material containing meltable components comprising fibers or polymeric binders including natural fibers such as cellulose, wood pulp, cotton, jute, hemp; synthetic fibers such as fiberglass, rayon, polyester, polyolefin, acrylic, polyamid, aramid, polytetrafluroethylene metal, polyimide; binders such as bicomponent high melt/low melt polymer, copolymer polyester, polyvinyl chloride, polyvinyl acetate/chloride copolymer, copolymer polyamide, materials comprising blends wherein some of the constituent materials are not meltable; air and vapour permeable materials including microporous films such as those supplied by EXXON Chemical Co., III, US under the designation EXXAIRE or those supplied by Mitsui Chemical Co., Japan under the designation ESPOIR NO; and monolithic breathable materials such as Hytrel™ available from DuPont and Pebax™ available from ELF Atochern, France.

The crotch panel 151 has two surfaces; one is an inner surface 310 and the other is an outer surface 320 as illustrated in FIG. 1. The inner surface 310 is the surface of the crotch panel 151 which is generally oriented toward the wearer when the garment 100 is worn. The inner surface 310 typically at least partially comes in contact with the wearer's skin during use of the garment 100. The outer surface 320 is the surface of the crotch panel 151 which is generally oriented away from the wearer when the garment 100 is worn, and at least partially toward another garment if another garment is worn. The crotch panel may be an assembly typically comprising a liquid pervious topsheet, a liquid impervious backsheet, and an absorbent core in order to acquire and store bodily discharges. The crotch panel may further comprise other features added to form the composite absorbent assembly structure. Alternatively, the crotch panel may only comprise a liquid impervious sheet to, e.g., hold a supplemental inner pad inside.

In the embodiment shown in FIG. 1, the first waist panel 131, the second waist panel 141 and the crotch panel 151 comprise separate members from each other. The crotch panel 151 comprises a first end portion 301 including a fixed end 306 and a second end portion 302 including an openable end 305. The first end portion 301 is joined to the first waist panel 131 and the second end portion 302 is joined to the second waist panel 141 such that the garment 100 forms a pant shape when in use of the garment 100. The first end portion 301 overlaps with the first central segment 232 and the second end portion 302 overlaps with the second central 242. The fixed end 306 is permanently joined to the interior surface 131A of the first waist panel 131. The first end portion 301 overlapping with the first central segment 302 is also permanently joined to the interior surface 131A of the first waist panel 131. The openable end 305 is releasably joined to the interior surface 141A of the second waist panel 141 by the releasable joint 500. The second end portion 302 is also releasably joined to the interior surface 141A of the second waist panel 141 by the releasable joint 500. Thus, when the garment 100 is formed into a pant shape before use of the garment 100, the first end portion 301 is positioned inside the interior surface 131A of the first waist panel 131 and the second end portion 302 is positioned inside the interior surface 141A of the second waist panel 141 such that the inner surface 310 of the first end portion 131 and the inner surface 310 of the second end portion faces each other. This configuration of the garment 100 allows to employ the easy manufacturing process to form the garment as described hereinbelow. Further, such a configuration of the garment 100 allows the first and second end portions 301, 302 having the absorbent core 250 to sufficiently contact the wearer's body to effectively use the absorbent capacity of the absorbent core 250. After the releasable joint 500 is released, the second end portion 302 is displaced from the inside of the second waist panel 141 and openable with respect to the second waist panel 141. When the second end portion 302 is reclosed, the second end portion 302 is positioned outside the second waist panel 141 and is capable of being joined to the exterior surface 141B of the second waist panel 141 to form a pant shape of the garment 100. This allows easy operation of joining the second end portion 302 to the second waist panel 141 because joining operation can be made on the exterior surface 141B of the second waist panel 141. In an alternative embodiment, the crotch panel 151 may have opposed openable ends; one openable end being openable with respect to the second waist panel 141 and the other openable end being openable with respect to the first waist panel 131. The releasable joint may be provided adjacent to the opposite openable ends of the crotch panel and the fastening member may be provided adjacent to the opposite openable ends such that both of the opposite openable ends are openable and reclosable to both of the first and second waist panels.

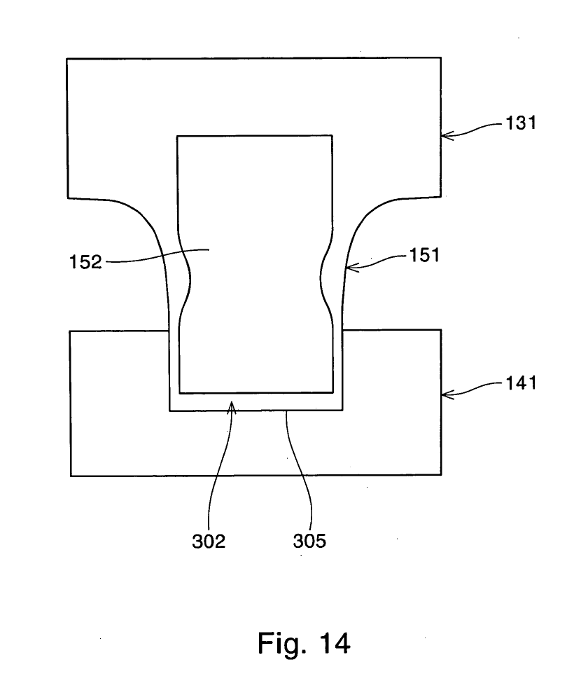

FIG. 14 shows a schematic view of an alternative embodiment of panel structures of the pant-type disposable garment. The garment 100 has a first waist panel 131, the second waist panel 141 and a crotch panel 151. The crotch panel 151 comprises an integral material of the first waist panel 131 instead of the separate crotch panel being permanently joined to the first waist panel 131. The crotch panel 151 has an openable portion 302 including an openable end 305 being openable and reclosable with respect to the second waist panel 141. In the embodiment shown in FIG. 14, an absorbent assembly 152 comprising a topsheet, a backsheet and an absorbent core may be disposed at least in the crotch panel 151.

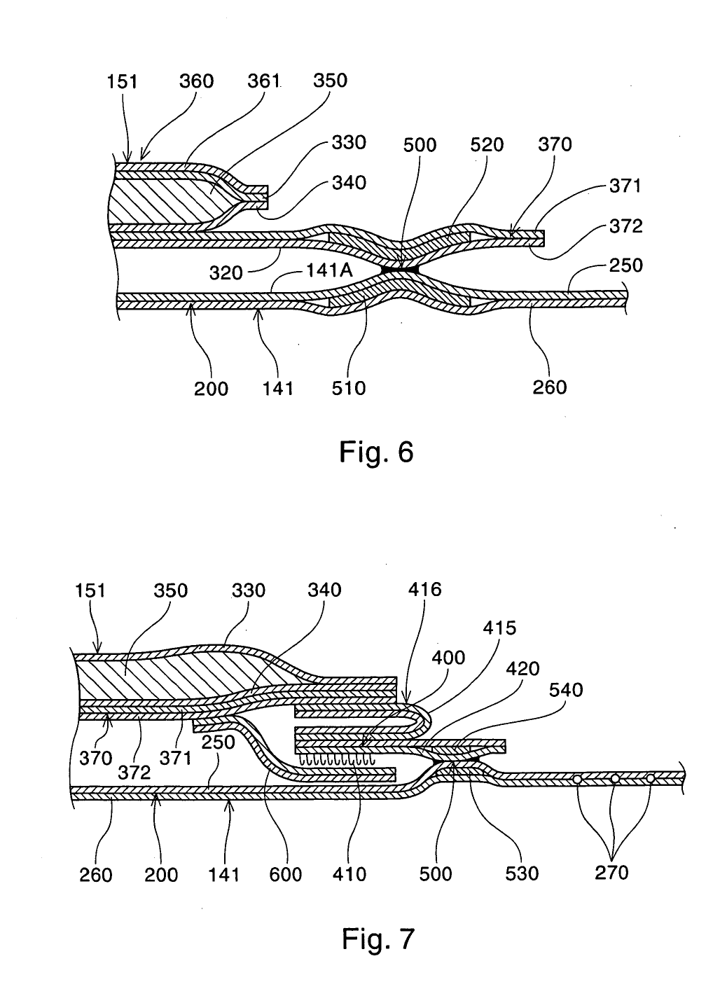

The crotch panel 151 comprises a liquid pervious topsheet 330, a liquid impervious backsheet 340, an absorbent core 350 positioned between the topsheet 330 and the backsheet 340. The crotch panel 151 preferably further comprises a pair of elasticized barrier cuffs 360. The topsheet 330 is preferably positioned so as to be adjacent to the inner surface of the absorbent core 350 and is preferably joined thereto and to the backsheet 340 in any suitable manner as is well known in the art. Such manners are described below with respect to joining the backsheet 340 to the absorbent core 350. The topsheet 330 and the backsheet 340 may be joined directly to each other. Alternatively, the topsheet 330 and the backsheet 340 may be indirectly joined together by directly joining them to other elements such as the absorbent core 350, the elasticized barrier cuffs 360 and the like in any suitable manner as is well known in the art. The backsheet 340 is preferably positioned so as to be adjacent to the outer surface of the absorbent core 350 and is preferably joined thereto in any suitable manner as is well known in the art. For example, the backsheet 340 may be secured to the absorbent core 350 by a uniform continuous layer of adhesive, a patterned layer of adhesive, or an array of separate lines, spirals, or spots of adhesive. Adhesives which have been found to be satisfactory are manufactured by H. B. Fuller Company of St. Paul, Minn. and marketed as HL-1258. Heat bonds, pressure bonds, ultrasonic bonds, dynamic mechanical bonds, or any other suitable means or combinations thereof as are known in the art may be used. While the topsheet 330, the backsheet 340, and the absorbent core 350 may be assembled in a variety of well known configurations in order to form a crotch panel 151, exemplary assembly configurations are described generally in U.S. Pat. No. 5,074,854 and International Patent Publication No. WO 90/04375. Further, as illustrated in FIGS. 6 and 7, the crotch panel 151 may further comprise an outermost layer 370 of nonwoven laminated on the outer surface of the backsheet 340 to provide the garment 100 with more cloth-like appearance in the crotch region 150 if the outer surface 320 of the crotch panel 151 is exposed. The outermost layer 370 typically comprises an inner nonwoven layer 371 and an outer nonwoven layer 372. In such an embodiment, any of the topsheet 330, the backsheet 340 and the absorbent core 350 may be omitted from the components of the crotch panel 151 if the garment 100 is intended to be used with another absorbent article such as a supplemental inner pad.

The topsheet 330 is adapted to contact the wearer's skin. The topsheet 330 faces the wearer while the garment 100 is worn, and is suitably employed to help isolate the wearer's skin from bodily discharges held in the absorbent core 350. Thus, the topsheet 330 is preferably compliant, soft feeling, and non-irritating to the wearer's skin. Further, the topsheet 330 may be less hydrophilic than the absorbent core 350 to present a relatively dry surface to the wearer. The topsheet 330 may also be liquid pervious for permitting liquid discharges (e.g., urine) to readily penetrate through its thickness. A suitable topsheet may be manufactured from a wide range of materials such as woven and nonwoven materials; polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be composed of natural fibers (e.g., wood or cotton fibers), synthetic fibers (e.g., polymeric fibers such as polyester, polypropylene, polyolefin, or polyethylene fibers), or a combination of natural and synthetic fibers. For example, the topsheet 330 may be composed of a meltblown or spunbonded web of polyolefin fibers. The topsheet 330 may also be a bonded-carded web composed of natural and/or synthetic fibers. Further, the topsheet 330 may be composed of a substantially hydrophobic material, and the hydrophobic material may optionally be treated with a surfactant or otherwise processed to impart a desired level of wettability and hydrophilicity. Such surfactants may be applied by any conventional means, such as spraying, printing, brush coating or the like. Surfactants may be applied to the entire topsheet 330 or may be selectively applied to particular sections of the topsheet 330, such as the medial section along the longitudinal centerline of the garment, to provide greater wettability of such sections. The topsheet 330 may further include a composition applied thereto that is configured to be transferred to the wearer's skin for improving the skin health of the wearer.

The backsheet 340 is to prevent bodily discharges absorbed and contained in the absorbent core 350 from wetting other articles which contact the garment 100 such as bed sheets and undergarments. Thus, the backsheet 340 is preferably impervious to liquids (e.g., urine), and is preferably manufactured from a thin plastic film although other flexible liquid impervious materials may also be used. As used herein, the term “flexible” refers to materials which are compliant and will readily conform to the general shape and contours of the human body. A suitable material for the backsheet 340 is a thermoplastic film having a thickness of from about 0.012 mm (0.5 mil) to about 0.051 mm (2.0 mils), preferably comprising polyethylene or polypropylene. If it is desired to present the backsheet 340 with a more cloth-like feel, the backsheet 340 may comprise a polyolefin film having a nonwoven web laminated to the exterior surface thereof, such as a spunbond web of polyolefin fibers. Further, the backsheet 340 may be formed of a woven or nonwoven fibrous web layer that has been totally or partially constructed or treated to impart a desired level of liquid impermeability to selected regions that are adjacent or proximate to the absorbent core 350. Still further, the backsheet 340 may be optionally composed of a micro-porous “breathable” material that permits vapors to escape from the garment 100 while still preventing liquid discharges form passing through the backsheet 340. For example, the backsheet 340 may include a vapor permeable nonwoven facing layer laminated to a micro-porous film. The backsheet 340 may also be an elastomeric material, such as a stretch-thermal laminate (“STL”), neck-bonded laminate (“NBL”), or stretch-bonded laminate (“SBL”) material. The backsheet 340 can also be embossed or otherwise provided with a matte finish to provide a more aesthetically pleasing appearance.

The absorbent core 350 may be any absorbent member which is generally compressible, conformable, non-irritating to the wearer's skin, and capable of absorbing and retaining liquids such as urine and other certain body discharges. The absorbent core 350 may be manufactured in a wide variety of sizes and shapes (e.g., rectangular, hourglass, “T”-shaped, asymmetric, etc.) and from a wide variety of liquid-absorbent materials commonly used in disposable diapers and the other absorbent articles such as comminuted wood pulp which is generally referred to as airfelt. Examples of other suitable absorbent materials include creped cellulose wadding; meltblown polymers including coform; chemically stiffened, modified or cross-linked cellulose fibers; tissue including tissue wraps and tissue laminates; absorbent foams; absorbent sponges; superabsorbent polymers; absorbent gelling materials; or any equivalent material or combinations of materials. Preferably, the absorbent core 350 includes superabsorbent particles and a carrier means for the superabsorbent particles. Such superabsorbent particles are typically manufactured from an absorbent gelling material. Preferably, the carrier means may be formed from comminuted wood pulp. Such comminuted wood pulp may be exchanged with synthetic, polymeric, meltblown fibers or with a combination of meltblown fibers and natural fibers. The configuration and construction of the absorbent core 350 may vary (e.g., the absorbent core may have varying caliper zones, a hydrophilic gradient, a superabsorbent gradient, or lower average density and lower average basis weight acquisition zones; or may comprise one or more layers or structures). Further, the size and absorbent capacity of the absorbent core 350 may also be varied to accommodate wearers ranging from infants through adults. However, the total absorbent capacity of the absorbent core 350 should be compatible with the intended use of the garment 100. The absorbent core 350 may further comprise an acquisition/distribution layer of chemically stiffened fibers and a fluid storage layer positioned underneath the acquisition/distribution layer.

The crotch panel 151 preferably further comprises a pair of elasticized barrier cuffs 360 for providing improved containment of liquids and other bodily discharges. The elasticized barrier cuff 360 typically comprises a barrier sheet 361 and an elastic spacing member 362. The elasticized barrier cuff 360 has a fixed edge 363 and a free edge 364. The fixed edge 363 is joined to the underlying component, such as the topsheet 330, the backsheet 340, the absorbent core 350, and the like, in any suitable manner as is well known in the art. The elastic spacing member 362 is enclosed by the barrier sheet 361 such that the elastic spacing member 362 is adjacent to the free edge 364 of the elastic barrier cuff 360. The elastic spacing member 362 allows the free edge 364 of the barrier cuff 360 to stand up away from the topsheet 330 in the crotch region 150 of the garment 100. While such an elasticized barrier cuff may comprise several different embodiments for reducing the leakage of bodily discharges, exemplary structures are described generally in U.S. Pat. No. 4,695,278; U.S. Pat. No. 4,795,454; and U.S. Pat. No. 5,685,874. The elasticized barrier cuffs 360 may be manufactured from a wide variety of materials such as polypropylene, polyester, rayon, nylon, foams, plastic films, formed films, and elastic foams. The barrier cuffs 360 may be woven, non-woven, spun-bonded, carded, or the like. A preferred barrier cuff comprises a polypropylene material containing no additional finish or surfactant to render it liquid impermeable.

As explained hereinabove, the leg opening 190 is provided with the elastic members 191. The elastic member 191 may be disposed along a portion of the side edge of the crotch panel 151 as shown in FIG. 1. However, the elastic member 191 may be disposed along the entire length of the side edge of the crotch panel 151.

The releasable joint 500 releasably joins the crotch panel 151 to the interior surface 141A of the second waist panel 141 to preform the pant shape of the garment 100. The releasable joint 500 is capable of being released to open the crotch panel 151 by intentional separation force exerted on the releasable joint 500 without causing serious damage to components of the garment 100. The releasable joint 500 may be provided to join a portion of the interior surface 141A of the second waist panel 141 and a portion of the outer surface 320 of the crotch panel 151 as shown in the embodiment shown in FIGS. 6 and 7, more concretely to join the interior layer 250 of the second waist panel 141 and the outermost layer 370 of the crotch panel 151. In the embodiment shown in FIGS. 1 and 3, the releasable joint 500 may comprise three components; one component extending in the lateral direction along and adjacent the openable end 305 of the crotch panel and the other two components extending in the longitudinal direction along and adjacent the side edge of the crotch panel 151. Alternatively, the releasable joint 500 may be formed with two or more of discrete components such as dots which extend intermittently in the lateral direction of the garment 100.

The releasable joint 500 may be formed any known means such as heat bonds, pressure bonds, ultrasonic bonds, cohesive bonds, adhesive bonds, perforations, or any other suitable means or combinations thereof. In the embodiment, the releasable joint 500 is provided between and joins the second waist panel 141 and the outermost layer 370 of the crotch panel 151. The second waist panel 141 is provided with an elastomeric layer 510 sandwiched the interior and exterior layers 250, 260, and the outermost layer 370 is provided with an elastomeric layer 520 sandwiched between the inner and outer nonwoven layers 371, 372. In the embodiment shown in FIG. 7, the releasable joint 500 is provided between and joins the second waist panel 141 and the substrate 420 of the fastening member 400 instead of the outermost layer 370 of the crotch panel 151. The second waist panel 141 is provided with an elastomeric layer 530 sandwiched between the interior and exterior layers 250, 260, and the substrate 420 comprises two nonwoven layers and an elastomeric layer 540 sandwiched between the nonwoven layers.

The releasable joint 500 is preferably formed to resist separation force that occurs during use and/or application of the garment 100. The term “separation force”, as used herein, refers to forces to separate one component of the garment 100 from another component of the garment 100 when such components are attached to each other. Separation force typically includes shear force and peel force. The term “shear force”, as used herein, refers to separation force acting generally parallel to the plane where the releasable joint 500 is formed. The term “peel force”, as used herein, separation force acting in the direction away from the wearer when the garment 100 is worn. The shear force to release the releasable joint 500 should not be too small since the components joined to each other by the releasable joint 500 undesirably separated from each other during use of the garment 100. It can results in serious leakage of bodily discharges which may cause soil of the wearer's clothes. In contrast, the peel force to release the releasable joint 500 should not be too great since it is difficult for users and/or caregivers to release the releasable joint 500 in order to use the garment 100 as a pant-type refastenable diaper. In such a case, if the user/caregiver forcibly tries to release the releasable joint 500, the components of the garment 100 may tear when the releasable joint 500 is released. The elastomeric layers 510, 520, 530 and 540 sandwiched between nonwoven layers allow the shear force and the peel force of the releasable joint 500 to be optimized. Suitable materials for the elastomeric layer can include synthetic rubber, natural rubber, thermoplastic olefinic elastomer, thermoplastic polyurethane elastomer, thermoplastic polyamide elastomer or the like. The shear force to release the releasable joint 500 is preferably greater than about 3.9 N/cm, more preferably ranges between about 5.9 N/cm and about 23.6 N/cm. The peel force to release the releasable joint 500 is preferably smaller than about 3.9 N/cm, more preferably ranges between about 0.079 N/cm and about 2.0 N/cm.

The following method describes the procedure for measuring the peel force and the shear force, in N/cm, to release the releasable joint 500. A suitable instrument used for the measurement of the peel force and the sear force to release the releasable joint 500 is INSTRON 5564 which may be equipped with either digital readout or strip chart data display for load and elongation. The following procedure is conducted under standard laboratory conditions at 23° C. (73° F.) and 50% relative humidity.
(1) Take a fresh garment from a package and cut a sample strip from the garment centered about the releasable joint. The sample strip comprises portions of the crotch panel and the waist panel having 25.4 mm (in the transverse direction) by 100 mm (in the longitudinal direction) size for each, and the portions should be releasably jointing each other. At least three (3) sample strips should be prepared for each measurement of the shear force and the peel force.
(2) Put the sample strip in the instrument. The way to set the sample strip is to insert one end of the portion taken from the crotch panel into the top clamp of the instrument first, and then to insert another end of the portion taken from the waist panel into the bottom clamp with enough tension to eliminate any slack of the sample strip.
(3) Pull the sample strip at a pulling speed of 500 mm/minute until completely separating the portion of crotch panel and the waist panel each other. This procedure is based on the International Standard ISO 527, which is incorporated herein by reference.
(4) Read the peak value of the shear or peel force value during the procedure (3).
(5) Repeat the above procedures (1) to (4) for the other sample strips.
(6) Calculate the Shear Force or the Peel Force as follows:
   Shear/Peel Force=Sum of the peak force values of the procedure (4) for samples tested/(N/cm) Number of test strips tested

The fastening member 400 is provided on and permanently fixed to the second end potion 302 of the crotch panel 151 adjacent to the openable end 305. The fastening member 400 is capable of refastening the second end portion 302 of the crotch panel 151 to the exterior surface 141B of the second waist panel 141 to reform the pant shape of the garment 100 after the second end portion 302 of the crotch panel 151 is displaced from the inside of the second waist panel 141 by releasing the releasable joint 500.

In a preferred embodiment, the fastening member 400 comprises one or more patches of hook-type material which is complementary to the second waist panel 141. In that case, the second waist panel 141 typically comprises receiving material complementary to the hook-type material of the fastening member 400. Alternatively, the fastening member 400 may comprise one or more patches of receiving material if the second waist panel 141 comprises hook-type material complementary to the receiving material of the fastening member 400. The term “hook-type material”, as used herein, refers to any material having a fastening system joined to and projecting from a substrate. The fastening system may have one or more mechanical engaging means which project, typically radially, from a shank which is joined to the substrate. The engaging means is typically the portion of the hook-type material which penetrates and is secured to the exposed surface of the complementary receiving material. Suitable hook-type material is sold by the Minnesota Mining and Manufacturing Company of Minneapolis, Minnesota as Model Number XPO-0040 and by Velcro U.S.A., Inc. of Manchester, N.H. as Hook 88. The term “receiving material”, as used herein, refers to any material having an exposed surface with tightly spaced openings complementary to the hook-type material. Such openings are typically defined by one or more strands or fibers. The complementary hook-type material may be entrapped by the exposed surface of the receiving material, and may not be withdrawn without interference. Suitable materials for such receiving material can include loop material, nonwoven material or the like. Such receiving material may also comprise Model Number CX-780 sold by KURARAY Corp. For the embodiment described herein, loop material having a pile depth of about 0.8 millimeters works well as a receiving material. Hook-type material and receiving material are considered “complementary” if the openings between the strands or fibers are sized to allow at least one engaging means of the hook-type material to penetrate into the exposed surface of the receiving material and to be engaged or intercepted thereby.

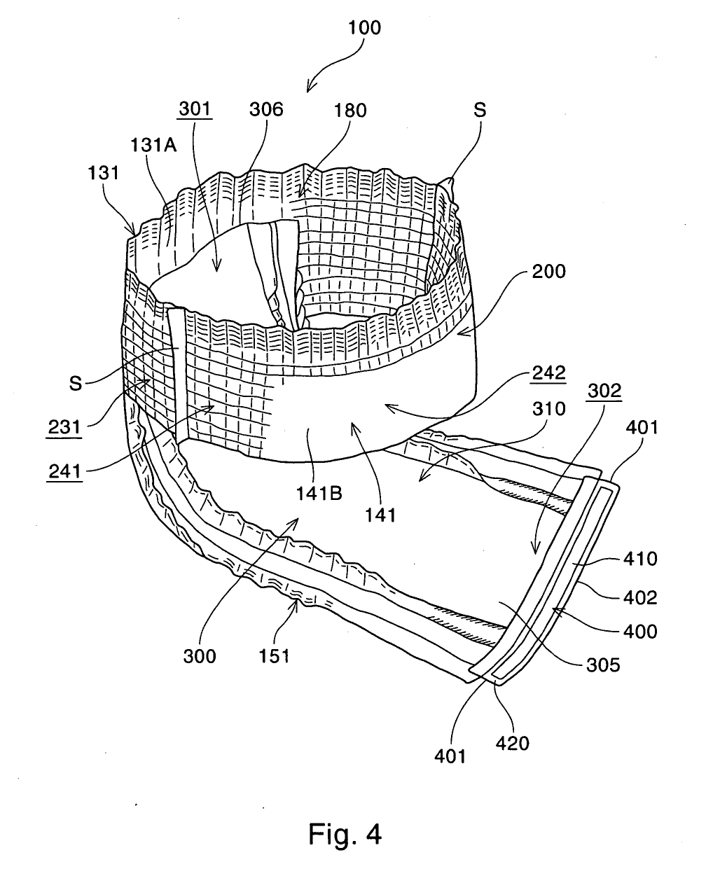

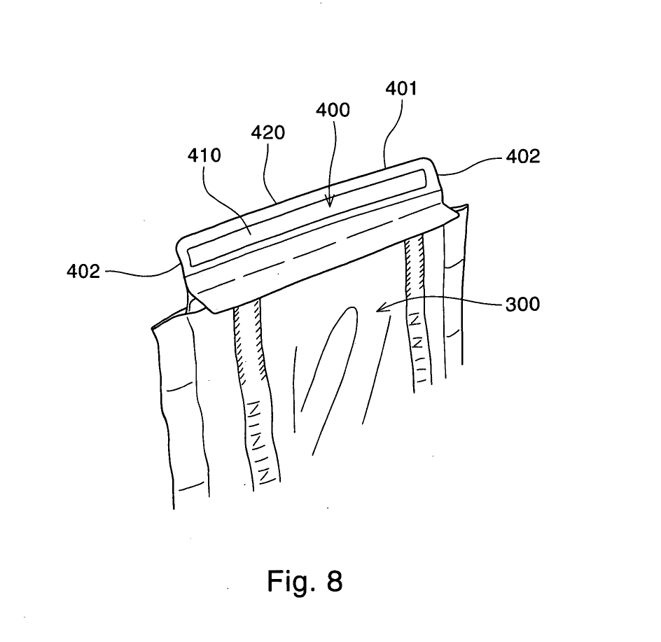

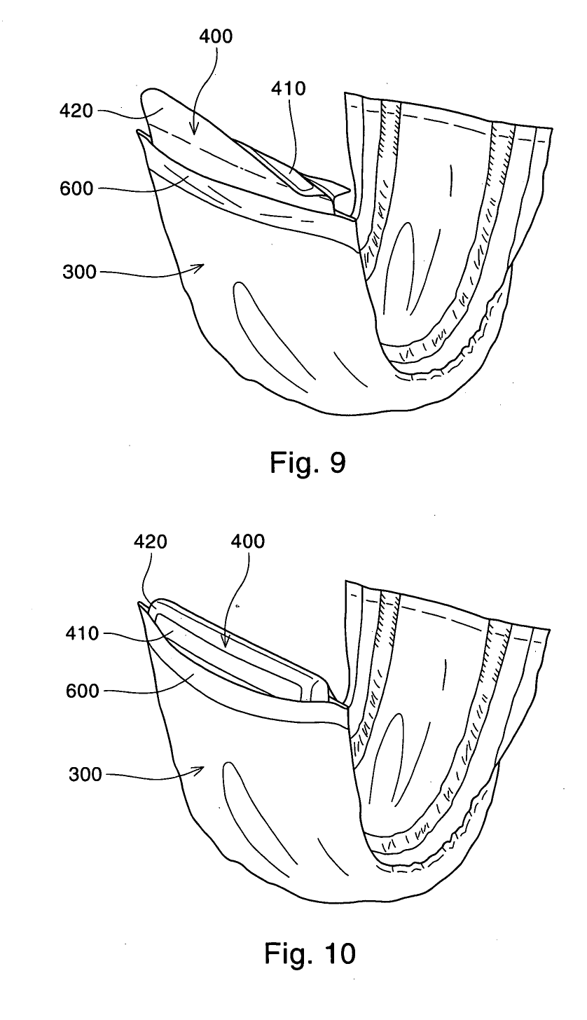

As illustrated in FIGS. 4, and 7 to 10, the fastening member 400 comprises a patch 410 of hook-type material which is complementary to the exterior surface 141B of the second waist panel 141, and a substrate 420 to which the patch 410 is attached. The patch 410 may be integral with the substrate 420, or may be a separate element attached to the substrate 420. The patch 410 may be spaced inwardly from the transverse side edges 401 and the longitudinal end edge 402 of the fastening member 400 at least approximately 0.1 centimeters, to provide for variations in positioning during manufacture, and to prevent the rough edge of the patch 410 from contacting and irritating the skin of the wearer. The patch 410 may be provided in any shape as long as the fastening member 400 is capable of refastening the crotch panel 151 to the second waist panel 141. Such suitable shapes include, but are not limited to: circle or oval shape; semicircle shape; sector shape; triangle shape; square, rectangular or diamond shape; a polygonal shape such as pentagon, hexagon, or the like; or any combination of the above. The patch 410 may be permanently bonded to the substrate 420 by any means well known in the art such that the joining strength exceeds the desired peel and shear strength. In another embodiment (not shown), the fastening member 400 may comprise one or more patches of adhesive instead of patches of hook or loop-type material. Suitable adhesive is sold by Eastman Chemical Products Company of Kingsport, Tenn. under the tradename Eastobond A-3. If adhesive patches are selected for the fastening member 400, the second waist panel 141 should be adapted to provide a complementary attachment surface to which such adhesive patches will readily adhere. A polyethylene material is suitable for providing the second waist panel 141 with such a complementary attachment surface.

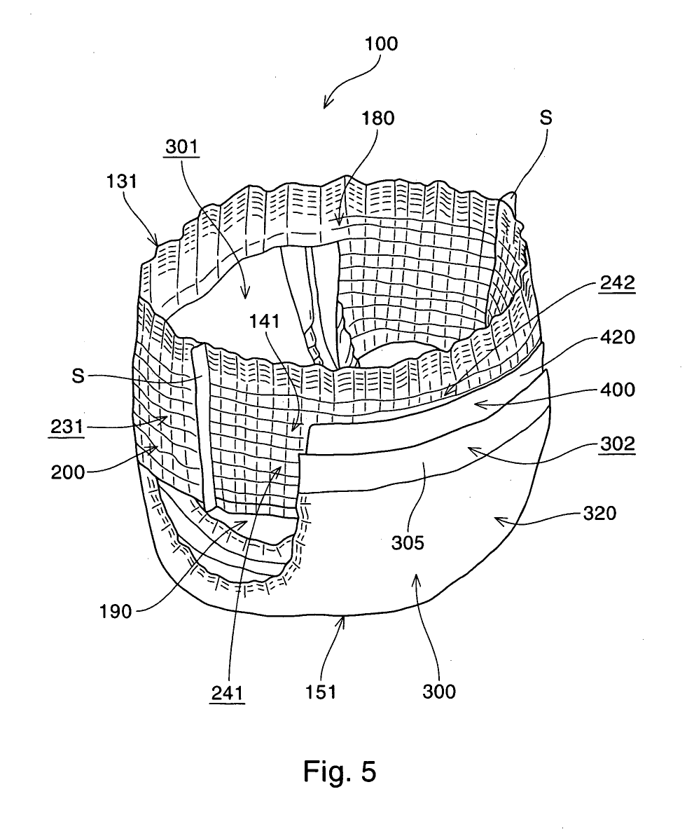

The fastening member 400 is positioned inside the second waist panel 141 and is not exposed outside of the second waist panel 141 until the releasable joint 500 is released in order to prevent undesirable contamination of the fastening member 400 before use. The fastening member 400 can be exposed by releasing the releasable joint 500 as illustrated in FIG. 4. Subsequently, the garment 100 can be refastened on the wearer's body by engaging the patch 410 of the fastening member 400 with the exterior surface 141B of the second waist panel 141 as illustrated in FIG. 5. The garment 100 may comprise an intermediate attachment member 415 to join the fastening member 400 to the crotch panel 151. In the preferred embodiment, the fastening member 400 and the intermediate attachment member 415 form a fastening member unit 416. Further, the garment 100 may comprise a holder 600 to contain the fastening member 400. As illustrated in FIGS. 7, 9 and 10, the fastening member 400 can be contained into the holder 600 so that the fastening member 400 is not exposed before use and is not fastened to any portion of the second waist panel 141 before the releasable joint 500 is released. In such an embodiment, the fastening member 400 can be protected from undesirable contamination and would not unexpectedly fasten to, e.g., a wearer's clothes when the crotch panel is reclosed through the wearer's legs.

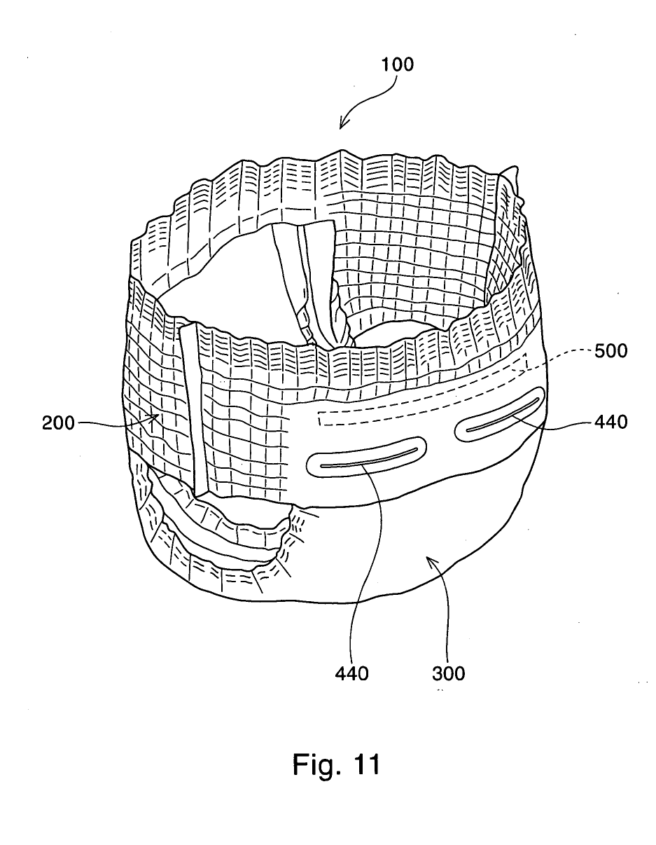

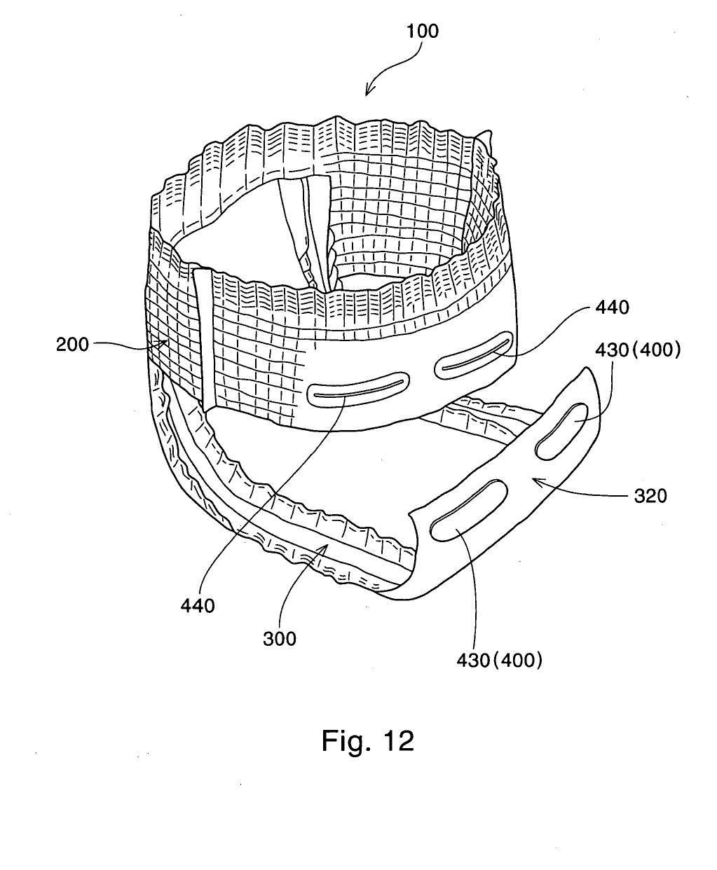

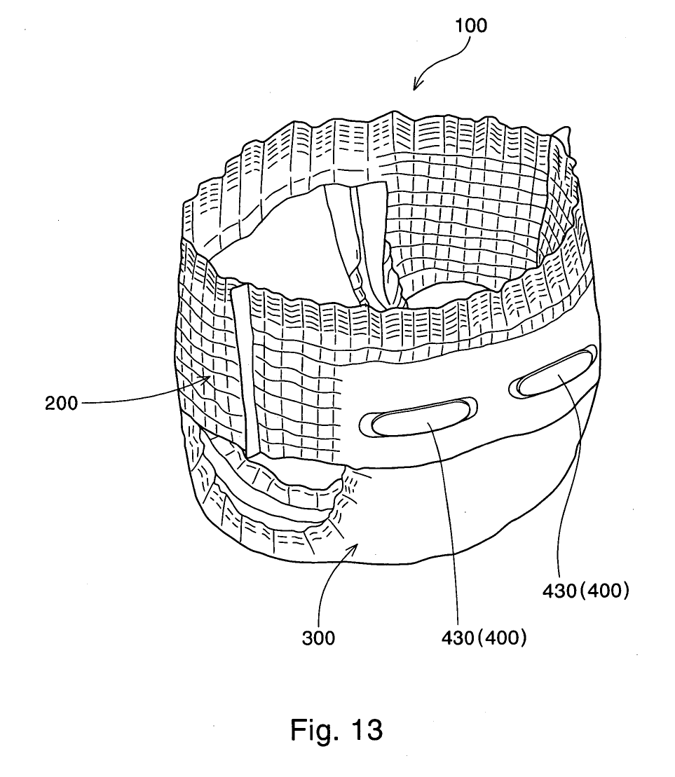

FIGS. 11 to 13 illustrate another embodiment of the fastening member 400. In the embodiment, the fastening member 400 comprises a tab member 430 fixed to the outer surface 320 of the crotch panel 300. If such a tab member 430 is selected for the fastening member 400, the second waist panel 141 can have a slot portion 440 with which the tab member 430 is releasably and repeatedly engageable. The tab member 430 may be also fixed to the inner surface 310 of the crotch panel 300. On the contrary, the fastening member 400 may have a slot portion if the second waist panel 141 comprises a tab member with which the slot portion is releasably and repeatedly engageable. As illustrated in FIG. 11, the tab member 430 of the fastening member 400 is positioned underneath the second waist panel 141 and is not exposed outside of the second waist panel 141 until the releasable joint 500 is released. The tab member 430 of the fastening member 400 can be exposed by releasing the releasable joint 500 as illustrated in FIG. 12. Subsequently, the garment 100 can be refastened on the wearer's body by passing the tab member 430 through the slot portion 440 as illustrated in FIG. 13. Any other known fastening means e.g., cohesive, magnets, hermaphroditic fasteners, buckles, buttons, clasps or the like, may be also acceptable for the fastening member 400.

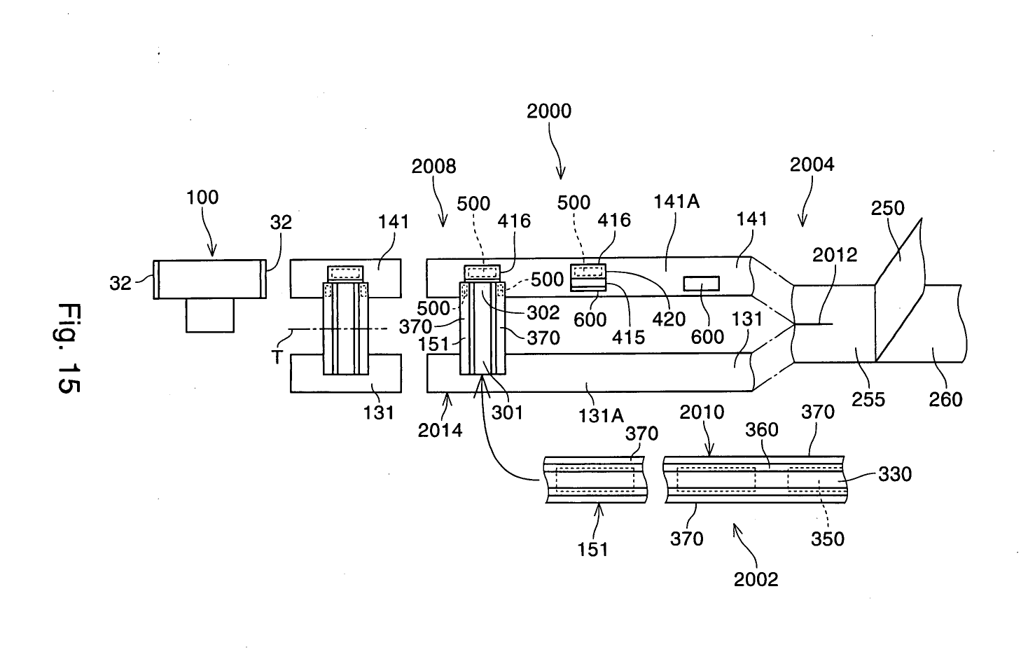

FIG. 15 shows a schematic view to explain the process for forming the garment 100. Since FIG. 15 is a schematic view, it should be noted that various parts of the garment have been omitted, such as the elastic material and the leg elastic material. The process 2000 primarily comprises three sections; a crotch panel forming section 2002, a waist panel forming section 2004 and an assembly section 2008.

The crotch panel forming section 2002 combines elements forming the crotch panel 151 such as the topsheet 330, the backsheet 340 (not shown in FIG. 15), the absorbent core 350 and the barrier cuffs 360 such that the absorbent core 350 is sandwiched between the topsheet 330 and the backsheet 340. The outermost layer 370 such as a nonwoven layer is joined to the backsheet 340 and the leg elastic member 191 (not shown in FIG. 15) is sandwiched between the backsheet 340 and the outermost layer 370. These elements are joined to each other by any known means such as adhesives or heat bonding to form an intermediate crotch panel assembly 2010. The intermediate crotch panel assembly 2010 is then cut into the individual crotch panel 151. The individual crotch panel 151 is turned by 90 degree and fed into the assembly section 2008.

The waist panel forming section 2004 combines the exterior layer web 260 and the interior layer web 250 to form a continuous layer web 255. When joining the exterior layer web 260 and the interior layer web 250, the elastic strands 270 (not shown in FIG. 15) is sandwiched therebetween. The continuous layer web 255 is cut along a cut line 2012 such as a straight cut line, a curved cut line or a zigzag cut line to form a continuous first waist panel 131 and a continuous second waist panel 141. Then the continuous first and second waist panel webs 131, 141 are separated from one another.

The assembly section 2008 combines the individual crotch panel 151 with the continuous first and second waist panel webs 131, 141. Before joining the crotch panel 151 with the first and second panel webs 131, 141, the holder 600 and the fastening member unit 416 comprising the fastening member 400 (not shown in FIG. 15) and the intermediate attachment member 415 (refer to FIG. 7) are placed on the interior surface 141A of the second waist panel web 141. The holder 600 may be free from attachment to the interior surface 141A. Alternatively, the holder 600 may be provisionally joined on the interior surface 141A such that the holder 600 is not displaced while the holder 600 is conveyed and such that the provisional joint between the holder 600 and the interior surface. 141A can be readily released when the crotch panel 151 is opened from the second waist panel 141. The fastening member unit 416 may be assembled before placed on the interior surface 141A. The fastening member unit 416 is placed such that the fastening member 400 is covered by the holder 600. The substrate 420 of the fastening member unit 416 is then releasably joined to the interior surface 141A by the releasable joint 500 as explained hereinabove. The crotch panel 151 is placed on the continuous first and second waist panel webs 131, 141 at a predetermined interval to sandwich the holder 600 and the fastening member unit 416. The outer surface 320 of the second end portion 302 of the crotch panel 151 is permanently joined to the intermediate attachment member 415 and the holder 600 by any known means such as adhesive (refer to FIG. 7). The releasable joint 500 is again formed between the outermost layer 370 of the crotch panel 151 and the second waist panel 141 (refer to FIG. 6). The outer surface 320 of the first end portion 301 of the crotch panel 151 is permanently joined to the interior surface 131A of the first waist panel 131. The continuous garment assembly 2014 thus formed is cut into each individual garment 100. The individual garment 100 is then folded along the transverse centerline T in the crotch region and the first and second waist panels 131, 141 are joined at the seam 32 adjacent the side edges to form the waist opening and the leg openings. Thus, the first end portion 301 is positioned inside the first waist panel 131 and the second end portion is positioned inside the second waist panel 141 when the garment 100 is assembled before the garment 100 is used.

The process of forming the garment 100 shown in FIG. 15 is particularly useful in providing easy manufacturing process. The separate crotch panel 151 may be placed on and supported by the interior surface 131A, 141A of the first and second waist panels 131, 141 (i.e., first and second waist belts) so that a supporting mechanism for supporting the crotch panel 151 is not specifically required. When in use of the garment 100, the second end portion 302 of the crotch panel 151 may be opened by releasing the releasable joint 500. After the releasable joint 500 is released, the second end potion 302 is displaced from the inside of the second waist panel 141 and may be positioned outside the second waist panel 141. The second end portion 302 is capable of being joined to the exterior surface 141B of the second waist panel 141 by the fastening member 400 to form a pant shape of the garment. This allows easy operation of joining the second end portion 302 to the second waist panel 141 because joining operation can be made on the exterior surface 141B of the second waist panel 141. In the meantime, it would be possible to place the crotch panel 151 on the exterior surface 131B, 141B of the first and second waist panel 131, 141 instead of the interior surface 13 IA, 141A when manufacturing the garment. This could allow an easy manufacturing process and easy operation of joining the second end portion 302 to the second waist panel 14. However, it is not esthetically acceptable to the consumers since the first and second end portions 301, 302 of the crotch panel 151 are exposed outside. Further there would be higher risk of liquid leakage through the first and second end portions 301, 302 when the first and second end portions 301, 302 are exposed than when they are positioned inside the first and second waist panels 131, 141. Alternatively, it would be possible to place the first end portion 301 on the interior surface 131A of the first waist panel 131 and to place the second end portion 302 on the exterior surface 141B of the second waist panel 141 when manufacturing the garment. This could be done by firstly feeding the first waist panel 131, secondly placing the first end portion 301 of the crotch panel 151 on the interior surface 131A of the first waist panel 131 and subsequently feeding and placing the second waist panel 141 on the inner surface 310 of the second end portion 302 of the second waist panel 141. While such a garment would allow easy operation of the joining the second end portion 302 to the second waist panel 141 when in use of the garment, it would require a complicated manufacturing process since the second end portion 302 of the crotch panel 151 must be held in place not to get displaced until the second waist panel 141 is placed on the second end portion 302. Thus, the garment configuration of the present invention is the most effective and advantageous in operation of joining the second end portion 302 to the exterior surface 141B of the second waist panel 141 and in providing easy manufacturing process of the garment.

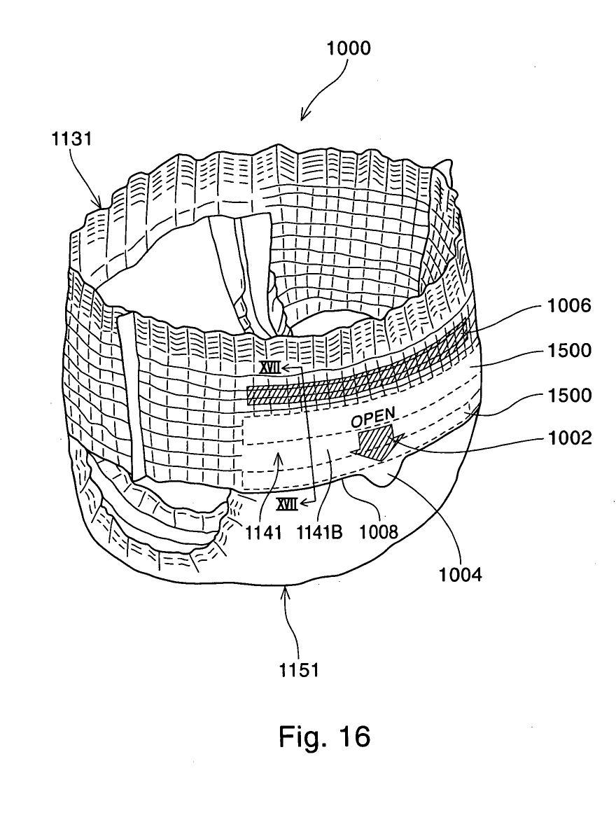

FIG. 16 illustrates another embodiment of the garment. The garment 1000 comprises a first waist panel 1131, a second waist panel 1141 and a crotch panel 1151. The garment 1000 has an opening indicator 1002 provided on the exterior surface 1141 B of the second waist panel 1141 and an opening tab 1004 for facilitating the opening operation. The garment 1000 also has a landing zone indicator 1006 provided on the second waist panel 1141 for fastening the fastening member 1400. Further, in the embodiment shown in FIG. 16, the releasable joint 1500 comprises two components; one component extending in the lateral direction along and adjacent the openable end 1305 of the crotch panel 1151 and the other component extending in the lateral direction along and adjacent the lower end 1008 of the second waist panel 1141. The other component of the releasable joint 1500 extending along and adjacent the lower end 1008 is positioned in the area where the absorbent core exits when the garment 1000 includes the absorbent core.

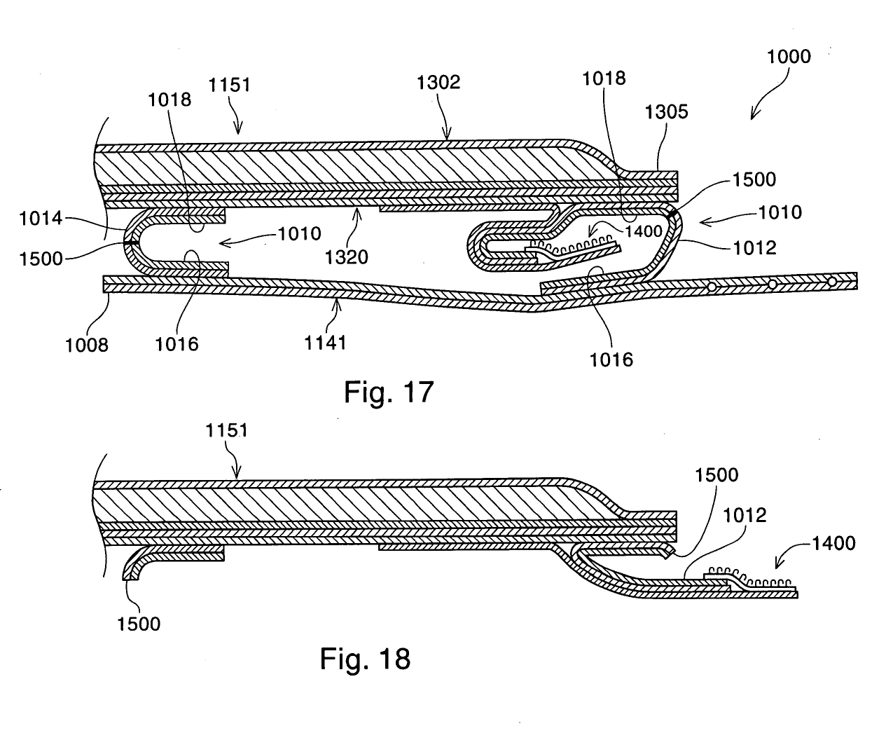

FIG. 17 is a cross-sectional view taken along line XVII-XVII of FIG. 16. The garment 1000 comprises an attachment unit 1010 provided between the second waist panel 1141 and the crotch panel 1151. The attachment unit 1010 comprises a first attachment unit 1012 provided along and adjacent the openable end 1305 of the crotch panel 1151 and a second attachment unit 1014 provided along and adjacent the lower end 1008 of the second waist panel 1141. Both of the first and second attachment units 1012 and 1014 are attached to the second waist panel 1141 at a first permanent attachment portion 1016 and are attached to the crotch panel 1151 at a second permanent attachment portion 1018. The fastening member 1400 is also provided along and adjacent the openable end 1305 of the crotch panel 1151 and joined to the crotch panel 1151. The fastening member 1400 is folded as shown in FIG. 17 until the releasable joint 1500 is released. In order to keep the folding configuration of the fastening member 1400, a portion of the fastening member 1400 may be, for example, provisionally joined to a portion of the first attachment unit 1012. The releasable joint 1500 is provided between the first attachment portion 1016 and the second attachment portion 1018 such that the attachment unit 1012, 1014 separates at the releasable joint 1500 when the releasable joint 1500 is released. In the embodiment shown in FIG. 17, the releasable joint 1500 may be formed with perforations formed in the attachment unit 1012, 1014. After the releasable joint 1500 is released, the fastening member 1400 is unfolded to provide a fastening function as shown in FIG. 18.

The process for forming the garment 1000 shown in FIGS. 16 to 18 can be modified from the process shown in FIG. 15. Instead of placing the holder 600 and the fastening member unit 416 on the interior surface 141A in the assembly section 2008 in FIG. 15, the first and second attachment units 1012, 1014 and the fastening member 1400 are placed on the interior surface 141A of the second waist panel web 141, and the first and second attachment units 1012, 1014 are permanently joined thereto at the first attachment portions 1016. Then the individual crotch panel 151 is placed on the continuous first and second waist panel webs 131, 141 at a predetermined interval to sandwich the first and second attachment units 1012, 1014 and the fastening member 1400. The first and second attachment units 1012, 1014 and the fastening member 1400 are permanently joined to the outer surface 1320 of the second end portion 1302 of the crotch panel 1151.

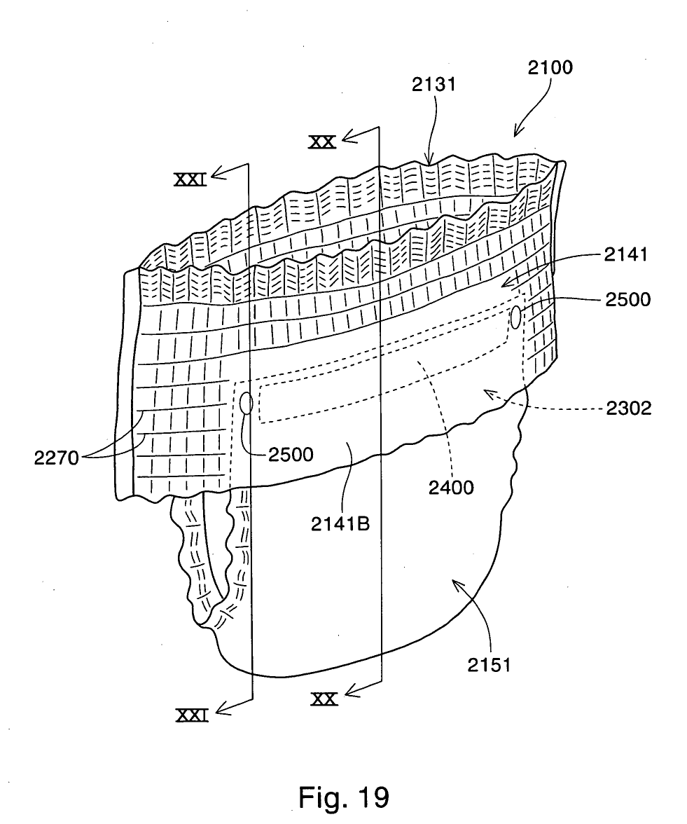

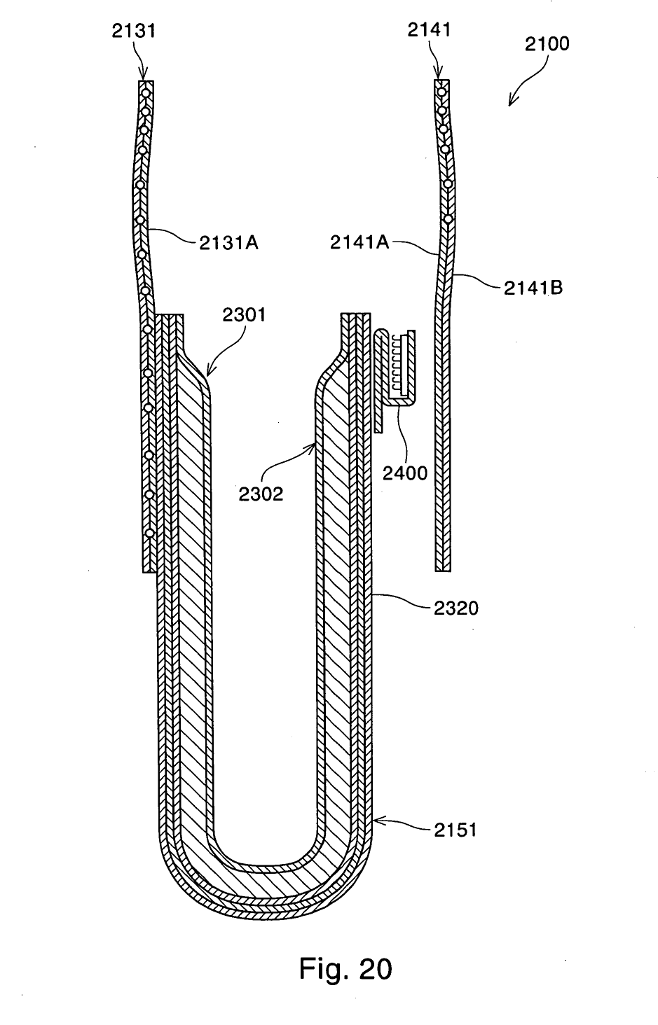

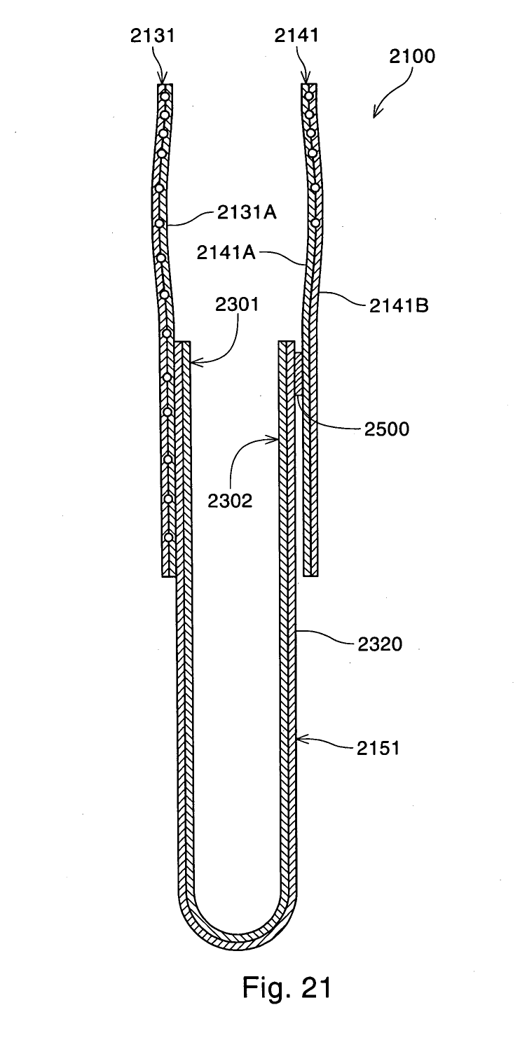

FIGS. 19 to 21 illustrate another embodiment of the garment. The first end portion 2301 of the crotch panel 2151 is positioned inside and permanently joined to the interior surface 213 IA of the first waist panel 2131 before use of the garment 2100. The second end portion 2302 of the crotch panel 2151 is positioned inside the interior surface 2141A of the second waist panel 2141 before use of the garment 2100. The garment 2100 comprises the folded fastening member 2400 provided on and permanently fixed to the outer surface 2320 of the second end potion 2302 of the crotch panel 2151. The garment 2100 also comprises a provisional joint 2500 instead of a releasable joint which is capable of resisting the separation force that occurs during use and/or application of the garment 100. The provisional joint 2500 may be positioned in the area where the absorbent core is not present as shown in FIG. 21. Alternatively, the provisional joint 2500 may be positioned in the area where the absorbent core is present. The provisional joint 2500 maintains the second end portion 2302 being positioned inside the second waist panel 2141 and the provisional joint 2500 is positioned between the outer surface 2320 of the second end portion 2302 and the interior surface 2141A of the second waist panel 2141 before use of the garment 2100. The second end portion 2302 is provisionally joined to the interior surface 2141A of the second waist panel 2141 only at the provisional joints 2500 as shown in FIGS. 20 and 21. The provisional joint 2500 is designed such that it is readily released by a force lower than the separation force that occurs during use and/or application of the garment 100. Therefore, the provisional joint 2500 is typically released before the garment 2100 is worn and the second end potion 2302 is joined to the exterior surface 2141B to form a pant shape of the garment by the unfolded fastening member 2400.

The process for forming the garment 2100 can be modified from the process shown in FIG. 15. Instead of placing the holder 600 and the fastening member unit 416 on the interior surface 141A in the assembly section 2008 in FIG. 15, the fastening member 2400 is placed on the interior surface 2141A of the second waist panel web 2141. Then the individual crotch panel 2151 is placed on the continuous first and second waist panel webs 2131, 2141 at a predetermined interval to sandwich the fastening member 2400. The fastening member 2400 is permanently joined to the outer surface 2320 of the second end portion 2302 of the crotch panel 2151. Before or after the fastening member 2400 is joined to the outer surface 2320 of the second end portion 2302, the provisional joint 2500 is formed between the outer surface 2320 of the second end portion 2302 and the interior surface 2141A of the second waist panel 2141 by any known means such as adhesive, heat bonding, pressure bonding, ultrasonic bonding or any combination thereof.

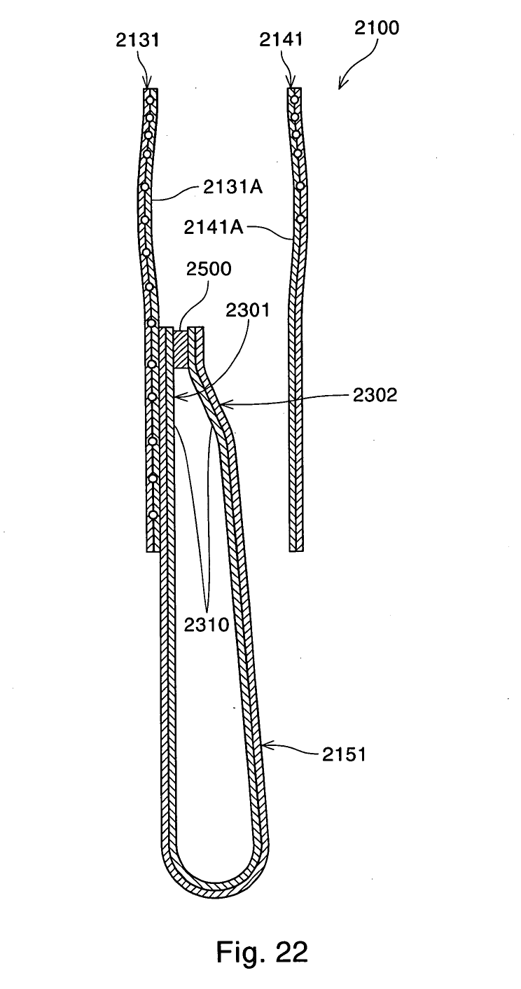

FIG. 22 illustrates another embodiment of the garment. The garment shown in FIG. 22 is different from the garment 2100 shown in FIGS. 19 to 21 only in the position of the provisional joint 2500. The provisional joint 2500 in FIG. 22 is positioned between the inner surface 2310 of the first end portion 2301 and the inner surface 2310 of the second end portion 2302 and maintains the second end portion 2302 being inside the second waist panel 2141 before use of the garment 2100.

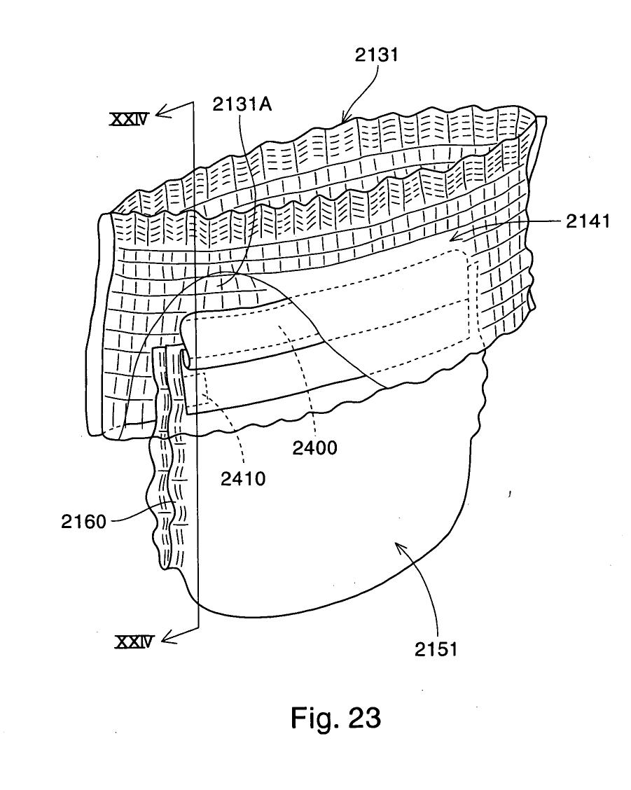

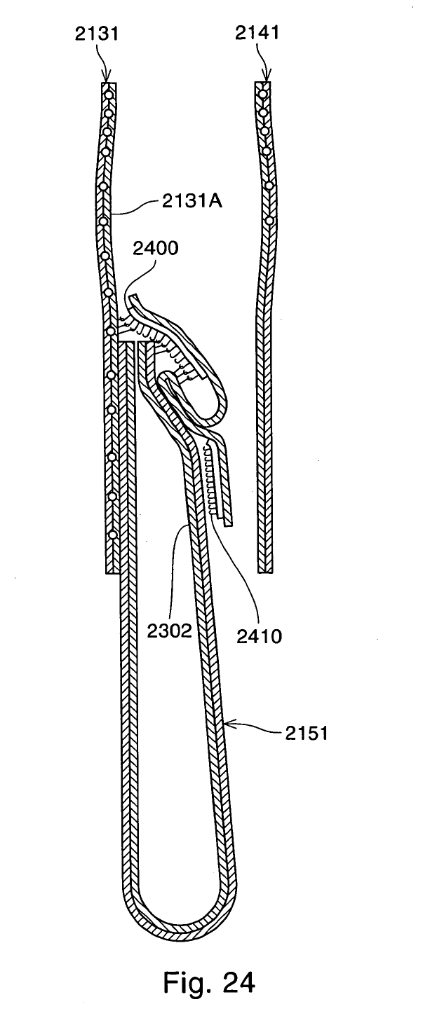

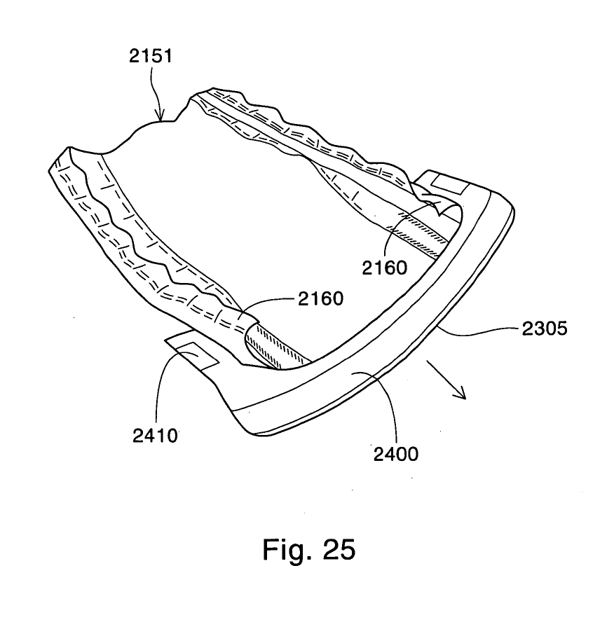

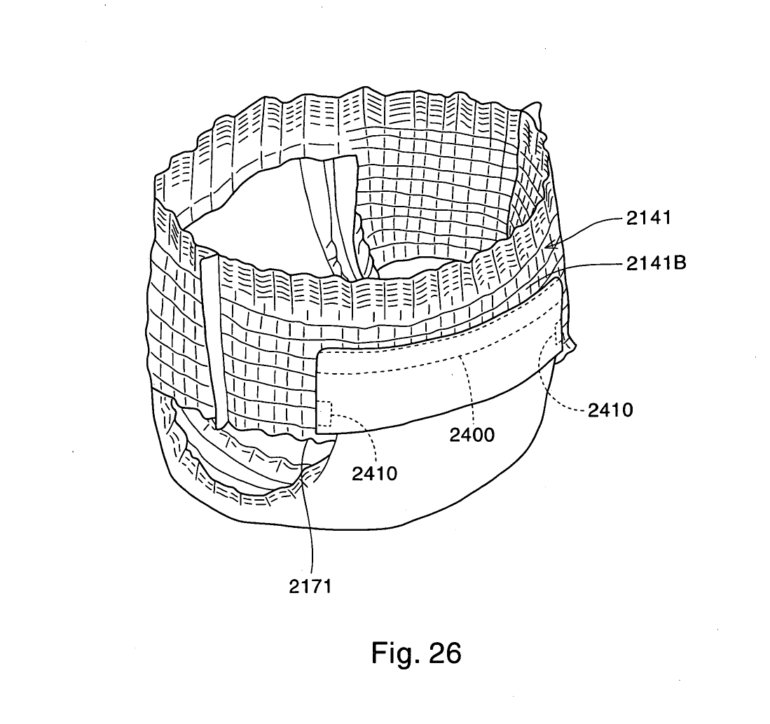

FIGS. 23 and 24 illustrate another embodiment of the garment. The garment shown in FIG. 24 is different from the garment shown in FIGS. 19 to 21 in the form of the provisional joint 2500. The provisional joint 2500 shown in FIG. 19 is formed by the provisional engagement of the fastening member 2400 to the interior surface 2131A of the first waist panel 2131 and maintains the second end portion 2302 being positioned inside the second waist panel 2141 before use of the garment 2100. The garment 2100 shown in FIG. 19 preferably comprises a pair of supplemental fastening member 2410. While the supplemental fastening members 2410 are provided inside the side edges 2160 of the crotch panel 2151, the side edges 2160 are turned up to expose the supplemental fastening member 2410 when the openable end 2305 is pulled as shown in FIG. 25. Then the fastening member 2400 and the supplemental fastening member 2410 are joined to the exterior surface 2141B as shown in FIG. 26. The supplemental fastening member 2410 helps to prevent the lower end edge 2171 of the second waist panel 2141 from getting turned up.

Alternatively, the garment may have neither of the provisional joint nor the releasable joint. Since at least one of the first and second waist panels 2131, 2141 has elastic strands 2270 on the side segment of the waist panel 2131, 2141 generating elasticity in the waist circumference direction of the garment, the second end portion 2302 of the crotch panel 2151 is maintained inside the second waist panel 2141 by the elasticity of the waist panel 2131, 2141.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A pant-type disposable garment having a waist opening and a pair of leg openings when in use of the garment, the garment comprising a first waist panel, a second waist panel and a crotch panel positioned between the first waist panel and the second waist panel, each of the first and the second waist panels having an interior surface orienting the wearer's skin and an exterior surface, the crotch panel comprising a first end portion joined to the first waist panel when in use of the garment and a second end portion joined to the second waist panel when in use of the garment, wherein
the first end portion of the crotch panel is positioned inside the first waist panel and the second end portion of the crotch panel is positioned inside the second waist panel before use of the garment, and
the second end portion of the crotch panel is capable of being joined to the exterior surface of the second waist panel to form a pant shape of the garment after the second end portion of the crotch panel is displaced from the inside of the second waist panel.

2. The pant-type disposable garment of claim 1 wherein each of the first waist panel, the second waist panel and the crotch panel comprises a separate member from each other.

3. The pant-type disposable garment of claim 2 wherein the first waist panel comprises a first waist belt member and the second waist panel comprises a second waist belt member.

4. The pant-type disposable garment of claim 1 wherein the garment comprises a fastening means provided on the second end portion of the crotch panel and being capable of fastening the second end portion of the crotch panel to the exterior surface of the second waist panel.

5. The pant-type disposable garment of claim 1 wherein the second waist panel is a front waist panel and the first waist panel is a rear waist panel.

6. The pant-type disposable garment of claim 1 wherein the second waist panel is a rear waist panel and the first waist panel is a front waist panel.

7. The pant type disposable garment of claim 1 wherein the first end portion is permanently joined to the first waist panel.

8. The pant-type disposable garment of claim 1 wherein the garment comprises a provisional joint to maintain the second end portion being positioned inside the second waist panel and the provisional joint is positioned between the second end portion of the crotch panel and the second waist panel.

9. The pant-type disposable garment of claim 1 wherein the garment comprises a provisional joint to maintain the second end portion being positioned inside the second waist panel and the provisional joint is positioned between the first end portion and the second end portion of the crotch panel.

10. The pant-type disposable garment of claim 1 wherein the garment comprises a releasable joint releasably joining the second end portion of the crotch panel to the second waist panel to preform a pant shape.
